(19) United States
(12) Patent Application Publication
(10) Pub. No.: US 2015/0327909 A1
(43) Pub. Date: Nov. 19, 2015

(54) TREATMENT APPARATUS

(71) Applicant: OLYMPUS CORPORATION, Tokyo (JP)

(72) Inventor: Kotaro Nakamura, Hachioji-shi (JP)

(73) Assignee: OLYMPUS CORPORATION, Tokyo (JP)

(21) Appl. No.: 14/812,638

(22) Filed: Jul. 29, 2015

Related U.S. Application Data
(63) Continuation of application No. PCT/JP2013/082976, filed on Dec. 9, 2013.

(30) Foreign Application Priority Data
Jan. 30, 2013 (JP) 2013-015343

(51) Int. Cl. A61B 18/14

(52) U.S. Cl. CPC A61B 18/14; A61B 2018/00095

(57) ABSTRACT

A treatment apparatus having an electrothermal conversion element, a heat transfer plate and a restricting structure arranged to be spaced apart from the heat transfer plate, wherein the restricting structure is configured to restrict a movement of a first part of a substrate of the electrothermal conversion element in a direction away from the heat transfer plate.

### CROSS REFERENCE TO RELATED APPLICATIONS

This application is a continuation of PCT International Application No. PCT/JP2013/082976, filed on Dec. 9, 2013, the entire content of which is .incorporated by this reference, and claims priority to Japanese Patent Application No. JP 2013-015343, filed on Jan. 30, 2013, the entire content of which is incorporated by this reference.

### BACKGROUND

1. Technical Field

The present invention relates to a treatment apparatus.

2. Background Art

There is generally known a treatment apparatus for treating body tissues by use of thermal energy. For example, Japanese Patent Application Laid-Open No. 2006-305236 Publication discloses therein a treatment apparatus described later. That is, in the treatment apparatus, a substrate for heat generation element is made of a metal member with excellent thermal conductivity. The substrate is formed with a heat generation part having a thin film resistance. The heat generation part is joined with a lead wire for supplying power. A filling agent is provided around a portion where the heat generation part and the lead wire are joined with each other, thereby securing an electric insulation property. The substrate for heat generation element has high thermal conductivity such that even if a body tissue partially contacts on such a heat generation element, preferable temperature controllability is secured. Therefore, the substrate is formed to be relatively thick by use of a member with higher thermal conductivity.

For example, in the treatment apparatus according to Japanese Patent Application Laid-Open No. 2006-305236 Publication described above, the heat generation part is provided to be stacked in the thickness direction of the heat generation element on the backside opposite to the surface of the substrate contacting with a body tissue, and the lead wire connected to the heat generation part is provided to be further stacked in the thickness direction of the heat generation element. On the other hand, in the treatment apparatus, thinning or downsizing in height of the heat generation element is required.

It is therefore an object of the present invention to provide a thinned treatment apparatus.

### SUMMARY

According to one aspect of the present invention, a treatment apparatus is provided. The treatment apparatus comprises: an electrothermal conversion element comprising: a substrate having a first part and a second part; and an electric resistance pattern arranged to at least the first part of the substrate, wherein the electric resistance pattern is configured to convert a first electric energy to heat; a heat transfer plate having: a first surface configured to contact a body tissue; and a second surface in thermal connection with the electrothermal conversion element, wherein the heat transfer plate is configured to conduct the heat from the second surface to the first surface, and wherein the second part of the substrate extends past and away from an end of the heat transfer plate; and a restricting structure arranged to be spaced apart from the second surface of the heat transfer plate, wherein the restricting structure is configured to restrict a movement of the first part of the substrate in a direction away from the second surface of the heat transfer plate.

### BRIEF DESCRIPTION OF THE DRAWINGS

FIG. 1 is a schematic diagram illustrating an exemplary structure of a treatment system according to each exemplary embodiment;

FIG. 2A is a schematic cross-section view illustrating an exemplary structure of a shaft and a holding part in an energy treatment tool according to each exemplary embodiment, which illustrates a state in which the holding part is closed;

FIG. 2B is a schematic cross-section view illustrating an exemplary structure of the shaft and the holding part in the energy treatment tool according to each exemplary embodiment, which illustrates a state in which the holding part is opened;

FIG. 3A is a schematic plan view illustrating an exemplary structure of a first holding member in the holding part according to each exemplary embodiment;

FIG. 3B is a schematic diagram illustrating an exemplary structure of the first holding member in the holding part according to each exemplary embodiment, which is a longitudinal cross-section view along the line 3B-3B illustrated in FIG. 3A;

FIG. 3C is a schematic diagram illustrating an exemplary structure of the first holding member in the holding part according to each exemplary embodiment, which is a transverse cross-section view along the line 3C-3C illustrated in FIG. 3A;

FIG. 4 is an exploded perspective view illustrating an exemplary structure of a first electrode part according to a first exemplary embodiment;

FIG. 5 is a perspective view illustrating an exemplary structure of a first high frequency electrode according to the first exemplary embodiment;

FIG. 6 is an enlarged perspective view illustrating an exemplary structure of a base end of the first high frequency electrode according to the first exemplary embodiment;

FIG. 7 is a plan view illustrating an exemplary structure of an electrothermal conversion element according to the first exemplary embodiment;

FIG. 8 is a plan view illustrating an exemplary structure of the first high frequency electrode according to the first exemplary embodiment;

FIG. 9 is a side view illustrating an exemplary structure of the first high frequency electrode according to the first exemplary embodiment;

FIG. 10 is a side view illustrating an exemplary structure of the base end of the first high frequency electrode according to the first exemplary embodiment;

FIG. 11 is a perspective view illustrating an exemplary structure of part of the first electrode part according to the first exemplary embodiment;

FIG. 12 is a perspective view illustrating an exemplary structure of part of the first electrode part according to the first exemplary embodiment;

FIG. 13 is a perspective view illustrating an exemplary structure of the first electrode part according to the first exemplary embodiment;

FIG. 14 is a perspective view illustrating an exemplary state in which first heater current lines and a first high frequency electrode current line are connected to part of the first electrode part according to the first exemplary embodiment;

FIG. 15 is a perspective view illustrating an exemplary structure of the first high frequency electrode according to a second exemplary embodiment; and

FIG. 16 is a perspective view illustrating an exemplary structure of part of the first electrode part according to the second exemplary embodiment.

### DETAILED DESCRIPTION

### First Exemplary Embodiment

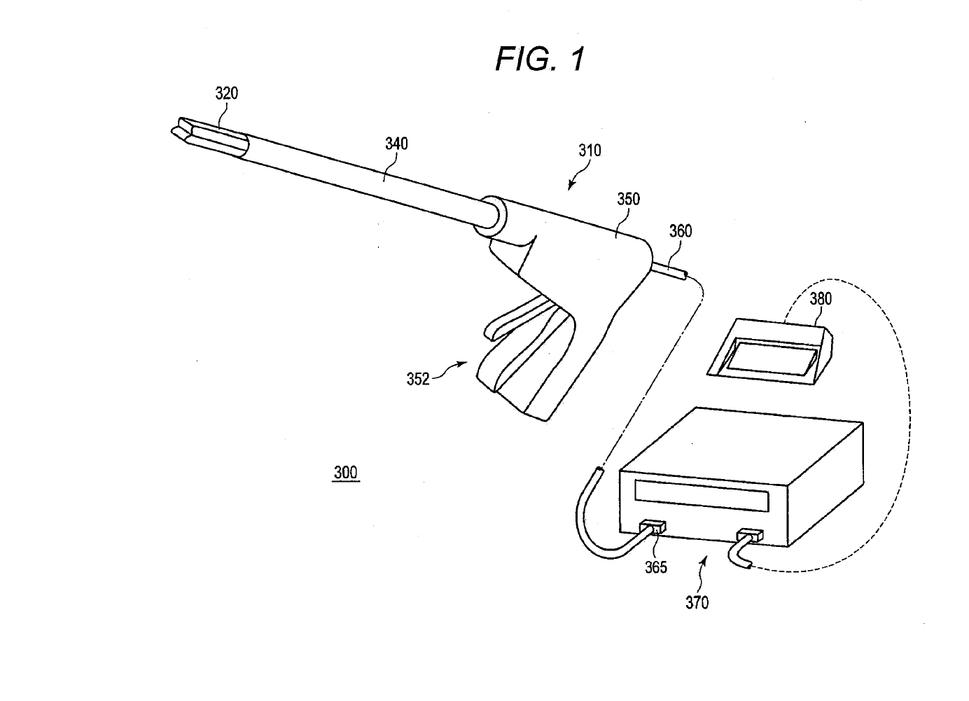

A first exemplary embodiment according to the present invention will be described with reference to the drawings. A treatment apparatus according to the present exemplary embodiment is used for treating body tissues. The treatment apparatus applies at least one of high frequency energy and thermal energy on body tissues. A treatment apparatus 300 is schematically illustrated in FIG. 1. As illustrated, the treatment apparatus 300 comprises an energy treatment tool 310, a control device 370, and a foot switch 380.

The energy treatment tool 310 is a linear type surgical treatment tool penetrating through the abdominal wall for treatment, for example. The energy treatment tool 310 includes a handle 350, a shaft 340 attached on the handle 350, and a holding part 320 provided on the tip end of the shaft 340. The holding part 320 is a treatment part which is openable and/or closable and is directed to perform treatments such as coagulation and incision of a body tissue by gripping the body tissue to be treated. For the following description, the side of the holding part 320 will be called tip end side and the side of the handle 350 will be called base end side. The handle 350 comprises a plurality of operation knobs 352 for operating the holding part 320. The handle 350 is further provided with a non-volatile memory (not illustrated) for storing therein eigenvalues and the like for the energy treatment tool 310. A shape of the energy treatment tool 310 illustrated herein is exemplary, and any other shape having the same function may be employed. For example, a forceps-like shape may be employed and the shaft may be curved.

The handle 350 is connected to the control device 370 via a cable 360. Herein, the cable 360 and the control device 370 are connected with each other via a connector 365, and the connection is removable to permit replacement of the energy treatment tool 310 in accordance with a treatment to be performed. The control device 370 is connected with the foot switch 380. The foot-operated foot switch 380 may be replaced with a hand-operated switch or another switch. An operator operates the pedal of the foot switch 380 thereby to switch ON/OFF energy supply from the control device 370 to the energy treatment tool 310.

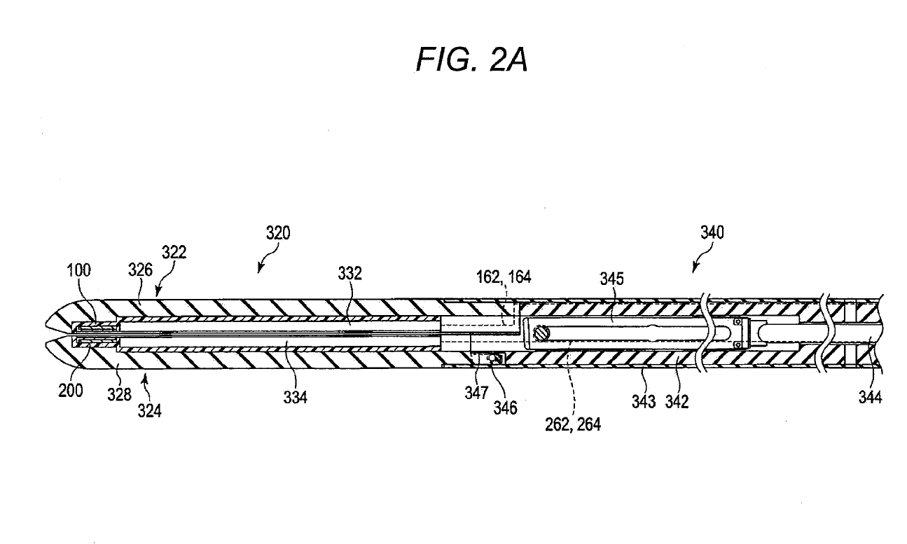

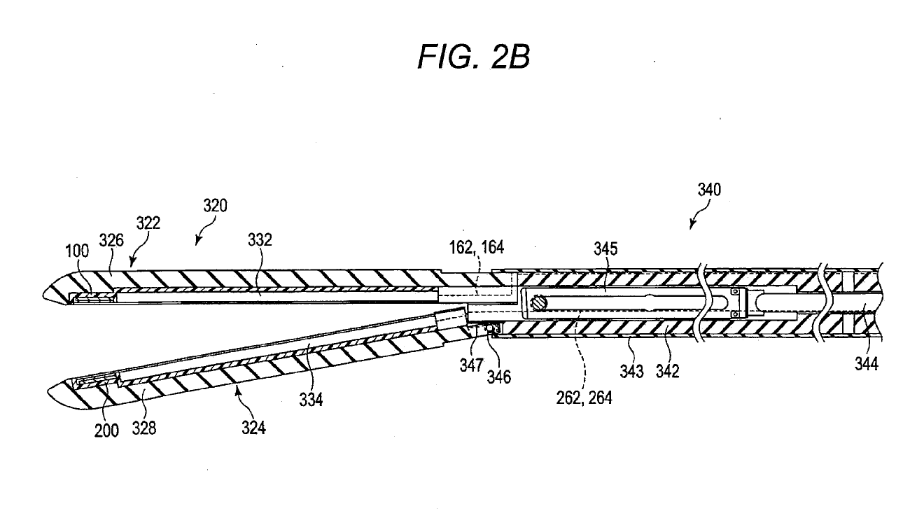

An exemplary structure of the holding part 320 and the shaft 340 is illustrated in FIG. 2A and FIG. 2B. FIG. 2A illustrates a state in which the holding part 320 is closed and FIG. 2B illustrates a state in which the holding part 320 is opened. The shaft 340 comprises a tube 342 and a sheath 343. The tube 342 is fixed at its base end to the handle 350. The sheath 343 is slidably arranged on the outer periphery of the tube 342 in the axial direction of the tube 342.

The holding part 320 is arranged on the tip end of the tube 342 . The holding part 320 comprises a first holding member 322 and a second holding member 324. The base of the first holding member 322 is fixed on the tip end of the tube 342 in the shaft 340. On the other hand, the base of the second holding member 324 is rotatably supported on the tip end of the tube 342 in the shaft 340 by a support pin 346. Therefore, the second holding member 324 axially rotates about the support pin 346 and opens/closes relative to the first holding member 322.

In a state in which the holding part 320 is closed, a cross-section shape in which the base of the first holding member 322 and the base of the second holding member 324 are put together is circular. The second holding member 324 is energized by an elastic member 347 such as plate spring to open relative to the first holding member 322. When the sheath 343 is slid toward the tip end of the tube 342 so that the base of the first holding member 322 and the base of the second holding member 324 are covered by the sheath 343, as illustrated in FIG. 2A, the first holding member 322 and the second holding member 324 are closed against an energizing force of the elastic member 347. On the other hand, when the sheath 343 is slid toward the base end side of the tube 342, as illustrated in FIG. 2B, the second holding member 324 is opened relative to the first holding member 322 due to an energizing force of the elastic member 347.

The tube 342 is inserted with a first high frequency electrode current line 162 connected to a first high frequency electrode 110 and a second high frequency electrode current line 262 connected to a second high frequency electrode 210, which will be described later. The tube 342 is inserted with a pair of first heater current lines 164 connected to an electrothermal conversion element 140 as a heat generation member described later arranged on the first high frequency electrode 110 and a pair of second heater current lines 264 connected to an electrothermal conversion element 230 arranged on a second high frequency electrode 210.

A drive rod 344 connected on its base end to one of the operation knobs 352 is movably arranged in the axial direction of the tube 342 inside the tube 342. A sheet-shaped cutter 345 forming a blade on its tip end is arranged on the tip end of the drive rod 344. When the operation knob 352 is operated, the cutter 345 is moved in the axial direction of the tube 342 via the drive rod 344. When the cutter 345 is moved toward the tip end, the cutter 345 is housed in a first cutter guide groove 332 and a second cutter guide groove 334 described later formed in the holding part 320.

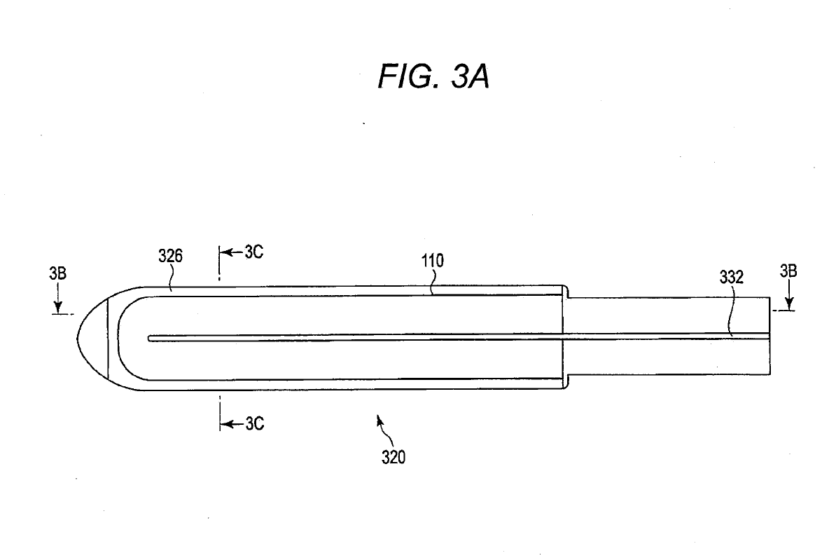

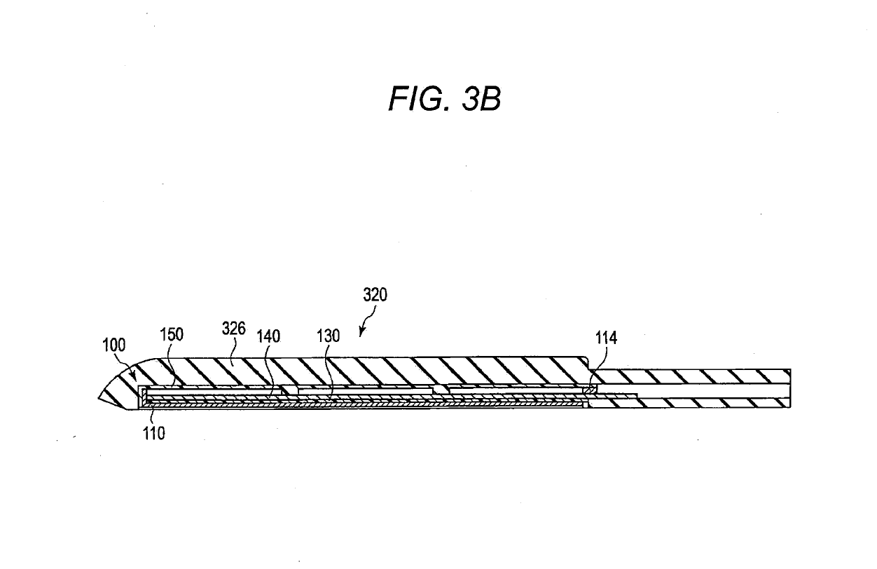

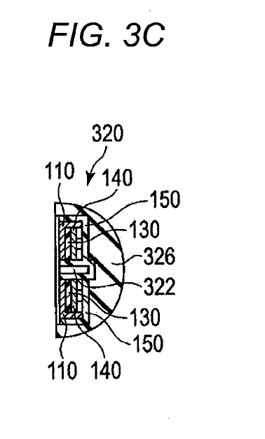

A structure of the first holding member 322 is schematically illustrated in FIG. 3A, FIG. 3B and FIG. 3C. As illustrated, the first holding member 322 is formed with the first cutter guide groove 332 for guiding the cutter 345. The first holding member 322 is provided with the first high frequency electrode 110 including a copper thin plate, for example. The first high frequency electrode 110 is configured to contact with a body tissue on either main surface thereof (which will be called first main surface below). The first high frequency electrode 110 includes the first cutter guide groove 332, and thus its planar shape is U-shaped as illustrated in FIG. 3A. The first high frequency electrode 110 is electrically connected with the first high frequency electrode current line 162 as described later in detail. The first high frequency electrode 110 is connected to the control device 370 via the first high frequency electrode current line 162 and the cable 360. The electrothermal conversion element 140 and a cover member 150 are arranged to a second main surface of the first high frequency electrode 110 which does not contact with a body tissue as described later in detail. A first electrode part 100 formed of the first high frequency electrode 110, the electrothermal conversion element 140, the cover member 150 and the like is formed in this way. The first electrode part 100 is embedded in and fixed on a first holding member main body 326. An exemplary structure of the first electrode part 100 will be described below in more detail.

As illustrated in FIG. 2A and FIG. 2B, the second holding member 324 is symmetrical in its shape to the first holding member 322, and has the same structure as the first holding member 322. That is, the second holding member 324 is formed with the second cutter guide groove 334 opposite to the first cutter guide groove 332. The second holding member 324 is provided with the second high frequency electrode 210 opposite to the first high frequency electrode 110. The second high frequency electrode 210 is configured to contact with a body tissue on either main surface thereof. The second high frequency electrode 210 is connected to the control device 370 via the second high frequency electrode current line 262 and the cable 360.

The electrothermal conversion element 230 and a cover member 250 are arranged to a surface of the second high frequency electrode 210 which does not contact with a body tissue. A second electrode part 200 formed of the second high frequency electrode 210, the electrothermal conversion element 230, the cover member 250 and the like is formed in this way. The second electrode part 200 is embedded in and fixed on a second holding member main body 328.

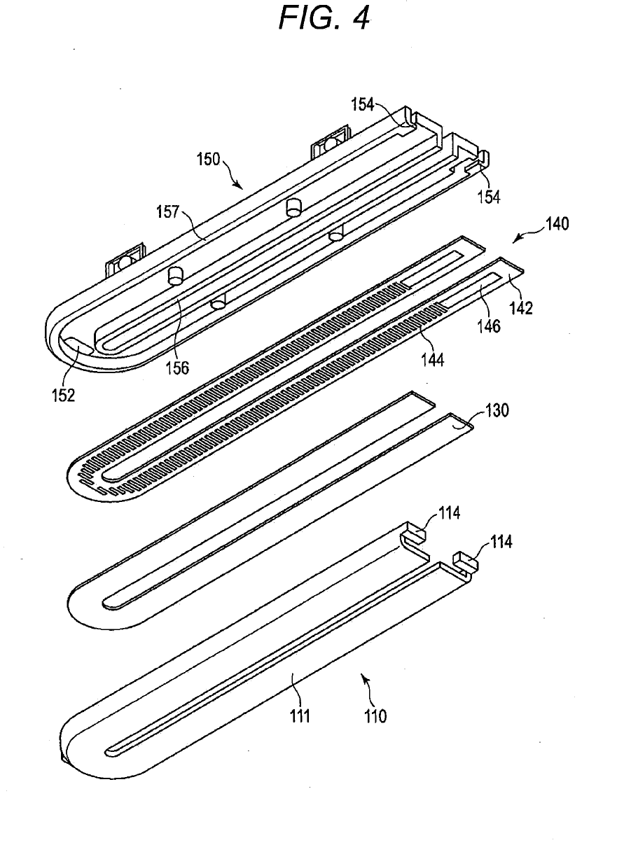

The first electrode part 100 will be described in detail. The second electrode part 200 has the same structure as the first electrode part 100, and thus the description of the second electrode part 200 will be omitted. An exploded perspective view of the first electrode part 100 is illustrated in FIG. 4. As illustrated, the first electrode part 100 includes the first high frequency electrode 110, a highly heat-conductive adhesive sheet 130, the electrothermal conversion element 140, and the cover member 150. The first high frequency electrode 110, the highly heat-conductive adhesive sheet 130, and the electrothermal conversion element 140 have a U shape to form the first cutter guide groove 332. The cover member 150 has a groove to form the first cutter guide groove 332.

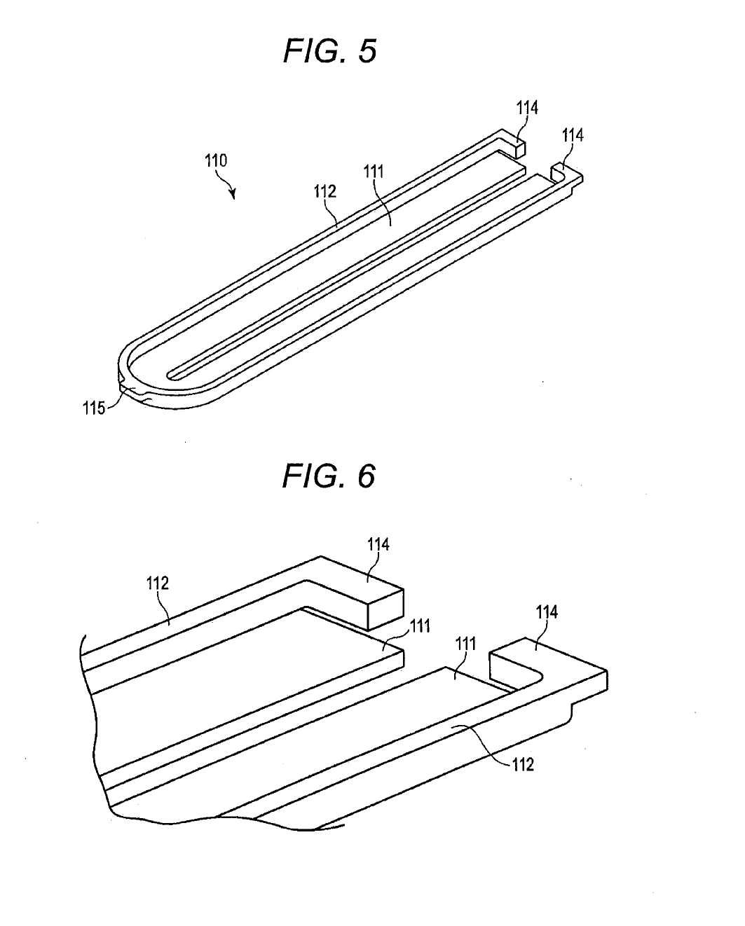

The first high frequency electrode 110 will be described with reference to FIG. 5 and FIG. 6. FIG. 5 is a perspective view of the first high frequency electrode 110, and FIG. 6 is an enlarged perspective view of the base end of the first high frequency electrode 110. The first high frequency electrode 110 is made of copper, for example, as described above. The first high frequency electrode 110 includes an electrode bottom 111 capable of contacting with a body tissue. A thickness of the electrode bottom 111 is around 0.5 mm, for example. A sidewall 112 is provided on the periphery of the electrode bottom 111 except the base end. The sidewall 112 is formed on the side of the cover member 150 to be perpendicular to the electrode bottom 111. Restricting structures 114 protruding from the sidewall 112 in the center axis direction of the first high frequency electrode 110 are provided at the base end of the first high frequency electrode 110. A convex part 115 protruding from the sidewall 112 is provided on the tip end of the first high frequency electrode 110.

The first high frequency electrode 110 may be formed by cutting work, for example. Further, the additionally-formed restricting structure 114 may be added to the sidewall 112 after the electrode bottom 111 and the sidewall 112 are formed. In this case, the electrode bottom 111 and the sidewall 112 may be formed also by bending work.

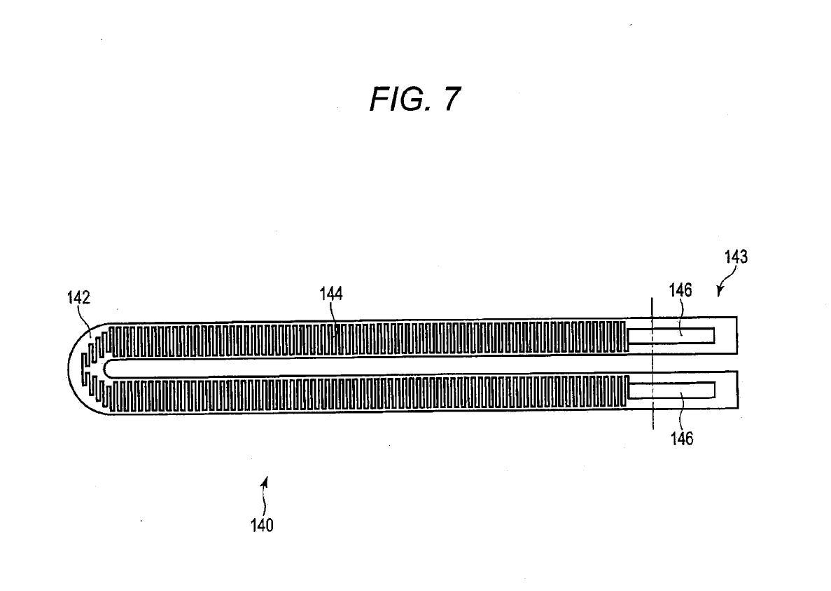

A plan view of the electrothermal conversion element 140 is illustrated in FIG. 7. As illustrated, the electrothermal conversion element 140 includes a substrate 142 made of polyimide, for example. The shape of the substrate 142 generally matches with the shape of the electrode bottom 111 of the first high frequency electrode 110, and the length thereof is slightly larger than that of the electrode bottom 111. A position corresponding to the end on the base end side of the electrode bottom 111 when the first electrode part 100 is assembled is indicated in a dashed-dotted line in FIG. 7. Specifically, when the first electrode part 100 is assembled, a first part of the substrate 142 is layered over and matches the shape of the electrode bottom 111 and second parts (also referred to as extension parts) 143 of the substrate 142 protrudes from the electrode bottom 111 past the dashed-dotted line in FIG. 7. An electric resistance pattern 144 is formed of a stainless (SUS) pattern, for example, in most of the substrate 142 except the extension parts 143. First lead connections 146 connected to both ends of the electric resistance pattern 144 are formed of a SUS pattern at the ends including the extension parts 143 of the substrate 142. When a voltage is applied to a pair of first lead connections 146, the electric resistance pattern 144 generates heat. In this way, the electrothermal conversion element 140 functions as a sheet heater. A thickness of the electrothermal conversion element 140 is around 100 μm, for example.

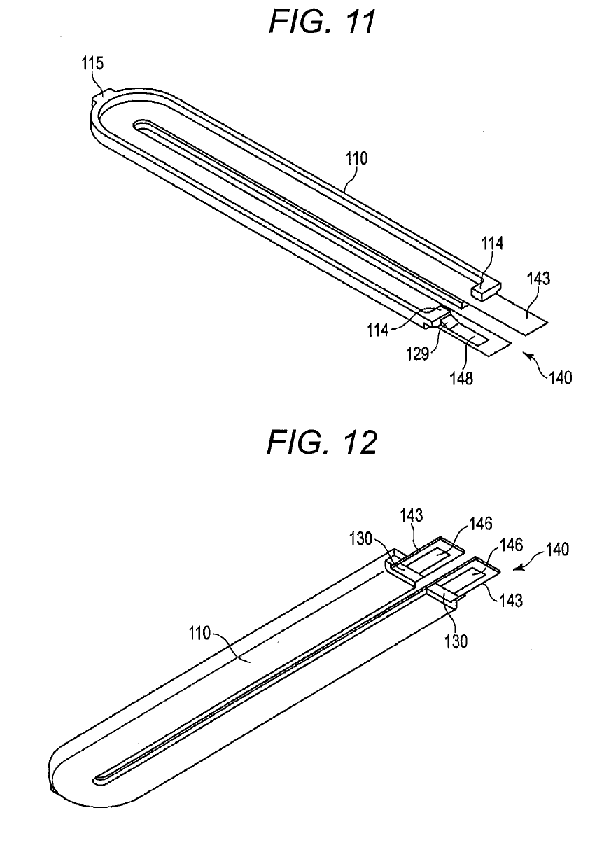

A second lead connection 148 is formed on a main surface opposite to the main surface of the substrate 142 on which the electric resistance pattern 144 is formed as illustrated in FIG. 11 described later. The second lead connection part 148 is configured to contact with the restricting structure 114 of the first high frequency electrode 110. The second lead connection 148 and the fixing part 114 are contacted with each other so that a voltage may be applied to the first high frequency electrode 110 via the second lead connection 148.

The electrode bottom 111 and the electrothermal conversion element 140 in the first high frequency electrode 110 are adhered to each other by the highly heat-conducive adhesive sheet 130. Herein, the electrothermal conversion element 140 is adhered with the surface forming the electric resistance pattern 144 thereon faced toward the first high frequency electrode 110. The highly heat-conductive adhesive sheet 130 is a sheet which is high in thermal conductivity and resistant to high temperature and has an adhesive property. The highly heat-conductive adhesive sheet 130 is made by mixing highly heat-conductive ceramic such as alumina or aluminum nitride with epoxy resin, for example. The highly heat-conductive adhesive sheet 130 has a high adhesive property, preferable thermal conductivity and an electric insulation property. A thickness of the highly heat-conductive adhesive sheet 130 is around 50 μm, for example.

The highly heat-conductive adhesive sheet 130 has substantially the same shape as the electrode bottom 111. The highly heat-conductive adhesive sheet 130 is slightly longer than the electrode bottom 111 of the first high frequency electrode 110. Since the highly heat-conductive adhesive sheet 130 is longer than the electrode bottom 111, an electric insulation property between the first high frequency electrode 110 and the first lead connections 146 is secured.

The cover member 150 is made of heat-resistant resin. The cover member 150 has a shape corresponding to the first high frequency electrode 110. A thickness of the cover member 150 is about 0.3 mm, for example. As illustrated in FIG. 4, a cutout 152 is provided on the tip end of the cover member 150. The cutout 152 engages with the convex part 115 of the first high frequency electrode 110. Further, convex parts 154 are provided on the base end side of the cover member 150. The convex parts 154 engage with the restricting structure 114 of the first high frequency electrode 110. In this way, the first high frequency electrode 110 and the cover member 150 are joined to each other via the engagements between the convex part 115 and the restricting structure 114 in the first high frequency electrode 110, and the cutout 152 and the convex parts 154 in the cover member 150. In this way, the first high frequency electrode 110 and the cover member 150 are arranged to each other in a simple structure, thereby decreasing manufacture cost. The cover member is provided with an inner wall 156 for forming the first cutter guide groove 332 and an outer wall 157 for covering the sidewall 112 of the first high frequency electrode 110.

In the structure of the first electrode part 100, the thickness of the first high frequency electrode 110 is relatively larger than those of other members. This is because thermal conductivity of the first high frequency electrode 110 is increased thereby to make a temperature of the first high frequency electrode 110 uniform even if a body tissue partially contacts on the first high frequency electrode 110. This is important in temperature control when a body tissue is anastomosed or joined by the energy treatment tool 310 according to the present exemplary embodiment.

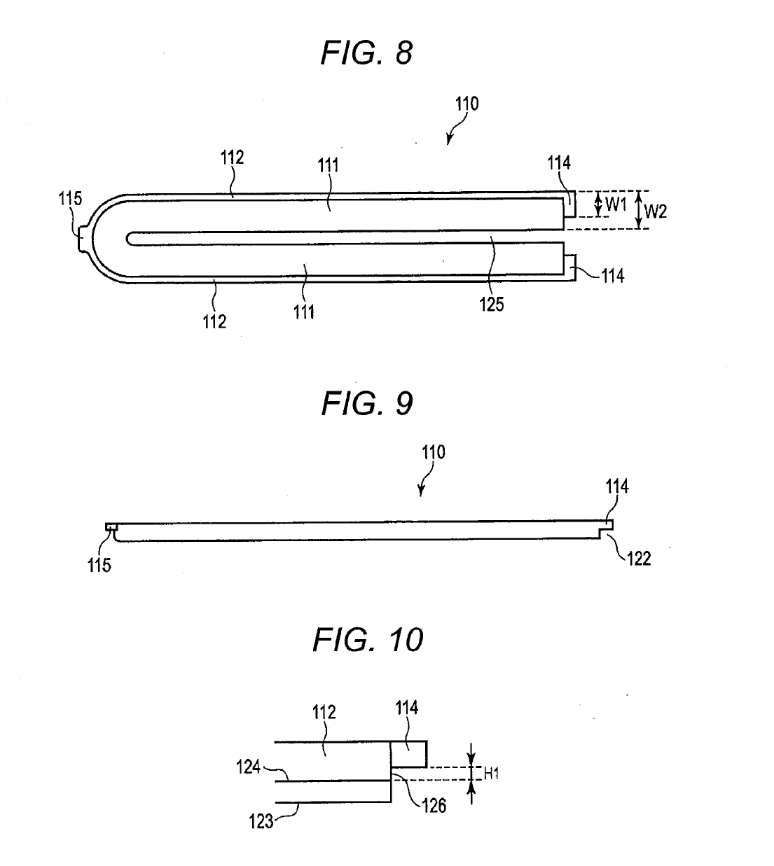

The first high frequency electrode 110 will be further described with reference to FIG. 8 to FIG. 10. FIG. 8 is a top view of the first high frequency electrode 110, FIG. 9 is a side view of the first high frequency electrode 110, and FIG. 10 is an enlarged side view of the first high frequency electrode 110 viewed from the inside of the sidewall 112. As illustrated in FIG. 8, the end faces on the tip end side of the restricting structures 114 and the end face on the base end side of the electrode bottom 111 are arranged on the same plane. Further, for example, the restricting structures 114 may be provided at the ends on the base end side of the electrode bottom 111, and thus the end faces on the tip end side of the restricting structures 114 may be arranged closer to the base end side than the end face on the base end side of the electrode bottom 111. That is, as viewed from above, a gap may be present between the electrode bottom 111 and the restricting structures 114. Still further, for example, the end faces on the tip end side of the restricting structures 114 may be arranged closer to the tip end side than the end face on the base end side of the electrode bottom 111. That is, as viewed from above, the electrode bottom 111 and the restricting structures 114 may be arranged to be overlapped on each other.

As illustrated in FIG. 8, in terms of either side across the cutout 125 forming the first cutter guide groove 332, it is preferable that a width W1 of the restricting structure 114 is larger than half of a width W2 of the first high frequency electrode 110. That is, W1>W2/2 is preferable. This is because the restricting structures 114 sufficiently restrict the electrothermal conversion element 140 as described later. The width W1 of the restricting structures 114 may be changed depending on stiffness of the electrothermal conversion element 140 as needed.

As illustrated in FIG. 9, concave parts 122 are provided on the electrode bottom 111 side of the restricting structures 114. The concave parts 122 engage with the convex parts 154 of the cover member 150.

As illustrated in FIG. 10, a surface contacting with a body tissue, out of the main surfaces of the electrode bottom 111, will be called first main surface 123, and a face forming the sidewall 112 thereon will be called second main surface 124. The restricting structures 114 are spaced apart from the second main surface 124 such that a height H1 of a gap 126 between the surface on the electrode bottom 111 side of the fixing part 114 and the second main surface 124 substantially matches with a total thickness of the highly heat-conductive adhesive sheet 130 and the electrothermal conversion element 140. The height of the gap 126 is around 150 μm, for example.

The second main surface of the electrode bottom 111 is attached to the electrothermal conversion element 140 by the highly heat-conductive adhesive sheet 130. Herein, the electrothermal conversion element 140 is arranged with the surface forming the electric resistance pattern 144 thereon faced toward the electrode bottom 111 as illustrated in FIG. 4. The base end of the electrothermal conversion element 140 is inserted into the gap 126 between the electrode bottom 111 and the restricting structures 114.

The perspective views where the electrothermal conversion element 140 is arranged in the first high frequency electrode 110 are illustrated in FIG. 11 and FIG. 12. As illustrated, the extension parts 143 of the electrothermal conversion element 140 protrude from the base end of the first high frequency electrode 110. As illustrated in FIG. 11, the second lead connection 148 provided on the extension part 143 contacts with the fixing part 114 of the first high frequency electrode 110. The second lead connection 148 and the fixing part 114 are fixed by conductive paste 129 so that electric conduction therebetween is secured. Connection by welding or soldering, for example, may be employed instead of the connection by the conductive paste 129. As illustrated in FIG. 12, the highly heat-conductive adhesive sheet 130 protrudes from the electrode bottom 111 thereby to secure insulation between the first high frequency electrode 110 and the first lead connections 146.

The first high frequency electrode 110 attached with the electrothermal conversion element 140 is fit with the cover member 150 as illustrated in FIG. 13. The first electrode part 100 is formed in this way.

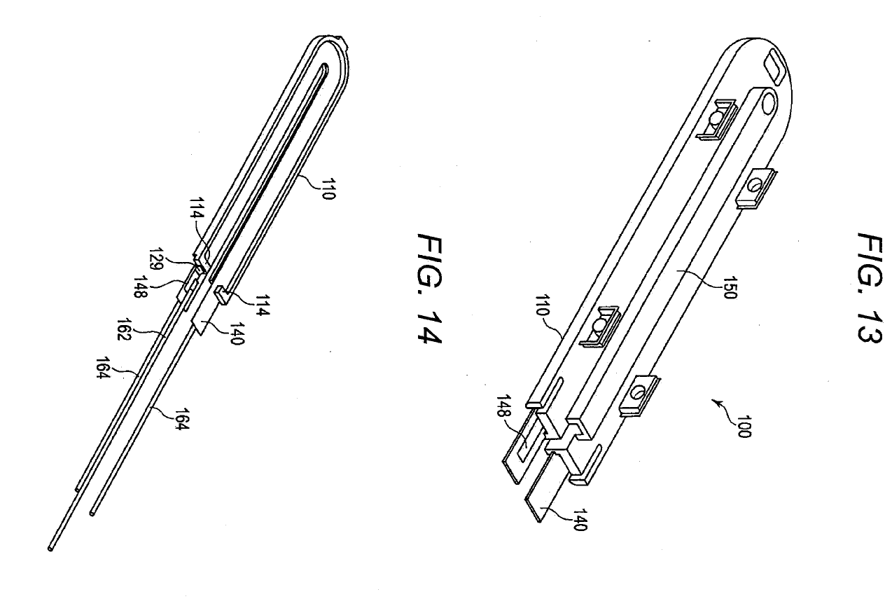

As illustrated in FIG. 14, the second lead connection 148 is connected with the first high frequency electrode current line 162 by soldering, for example. A high frequency voltage is applied to the first high frequency electrode 110 from the first high frequency electrode current line 162 via the second lead connection 148 so that the first high frequency electrode 110 applies a high frequency current to a body tissue gripped by the holding part 320. The first lead connections 146 are connected with the first heater current lines 164 by soldering, for example. A voltage is applied to the electric resistance pattern 144 from the first heater current lines 164 via the first lead connections 146 so that the electric resistance pattern 144 generates heat and the heat is transferred to the body tissue via the first high frequency electrode 110.

When the second lead connection 148 and the first high frequency electrode current line 162 are connected with each other and the first lead connections 146 and the first heater current lines 164 are connected with each other, the connection portions therebetween are preferably applied with a sealing agent made of silicon resin (not illustrated), for example.

The electric resistance pattern 144 of the electrothermal conversion element 140 is arranged closer to the first high frequency electrode 110 than to the substrate 142 of the electrothermal conversion element 140 with the highly heat-conductive adhesive sheet 130 intervened between the electric resistance pattern 144 and the first high frequency electrode 110. Thus, the electric resistance pattern 144 is thermally coupled with the first high frequency electrode 110 via the highly heat-conductive adhesive sheet 130. Only the highly heat-conductive adhesive sheet 130 is present between the electric resistance pattern 144 and the first high frequency electrode 110, and thus heat generated by the electric resistance pattern 144 is efficiently transferred to the first high frequency electrode 110.

In order to efficiently transfer heat generated by the electrothermal conversion element 140 to the first high frequency electrode 110, it is preferable that the cover member 150 and the first holding member main body 326 around the same have lower thermal conductivity than the first high frequency electrode 110 or the highly heat-conductive adhesive sheet 130. The cover member 150 and the first holding member main body 326 have low thermal conductivity so that loss of the heat generated by the electrothermal conversion element 140 is decreased.

The first electrode part 100 has been described above, and the second electrode part 200 is the same as the first electrode part 100.

The operations of the treatment apparatus 300 according to the present exemplary embodiment will be described below. The operator previously operates the input part of the control device 370 to set the output conditions of the treatment apparatus 300, such as setting power for high frequency energy output, target temperature for thermal energy output, and heating time. The treatment apparatus 300 may be configured such that the respective values are independently set or a set of setting values is selected depending on an operation.

The holding part 320 and the shaft 340 in the energy treatment tool 310 are inserted into the abdominal cavity via the peritoneum, for example. The operator operates the operation knobs 352 to open/close the holding part 320 so that a body tissue to be treated is gripped by the first holding member 322 and the second holding member 324. At this time, the body tissue to be treated contacts on the first main surfaces of both of the first high frequency electrode 110 provided on the first holding member 322 and the second high frequency electrode 210 provided on the second holding member 324.

When the body tissue to be treated is gripped by the holding part 320, the operator operates the foot switch 380. When the foot switch 380 is tuned ON, high frequency power for preset power is supplied from the control device 370 to the first high frequency electrode 110 and the second high frequency electrode 210 via the first high frequency electrode current line 162 passing inside the cable 360. The supplied power is on the order of 20 W to 80 W, for example. Consequently, the body tissue generates heat and the tissue is cauterized. The tissue modifies and coagulates due to the cauterization.

After the control device 370 stops outputting high frequency energy, the electrothermal conversion element 140 is supplied with power such that the temperature of the first high frequency electrode 110 reaches a target temperature. Herein, the target temperature is 200° C., for example. At this time, a current flows through the electric resistance pattern 144 of the electrothermal conversion element 140 from the control device 370 via the cable 360 and the first heater current lines 164. The electric resistance pattern 144 generates heat due to the current. The heat generated by the electric resistance pattern 144 is transferred to the first high frequency electrode 110 via the highly heat-conductive adhesive sheet 130. Consequently, the temperature of the first high frequency electrode 110 increases.

Similarly, the electrothermal conversion element 230 is supplied with power such that a temperature of the second high frequency electrode 210 reaches the target temperature. The electrothermal conversion element 230 in the second electrode part 200 is supplied with power from the control device 370 via the cable 360 and the second heater current lines 264 so that the temperature of the second high frequency electrode 210 increases.

The body tissue contacting with the first high frequency electrode 110 or the second high frequency electrode 210 is further cauterized and further coagulated by the heat. When the body tissue coagulates by the heating, the thermal energy stops being output. The operator finally operates the operation knobs 352 to move the cutter 345, thereby cutting the body tissue. The treatment of the body tissue is completed with the above operations.

As described above, for example, the first high frequency electrode 110 and the second high frequency electrode 210 function as a heat transfer plate configured to contact with a body tissue on a first main surface out of the first main surface and a second main surface, which are the front and back surfaces, and to transfer heat to the body tissue. For example, the electrothermal conversion element 140 functions as an electrothermal conversion element which is provided on the second main surface of the heat transfer plate, includes extension parts extending from the heat transfer plate, and forms with an electric resistance pattern for generating heat in response to an applied voltage and first lead connection parts connected to the electric resistance pattern and provided on the extension parts. For example, the restricting structures 114 function as restricting structures which are provided on ends where the electrothermal conversion element of the heat transfer plate extends, and grip the electrothermal conversion element between them and the heat transfer plate. For example, the first heater current lines 164 function as first lead wires which are electrically connected to the first lead connections 146 at the extension parts and supply the electric resistance pattern with power. For example, the second lead connection 148 functions as a second lead connection which is formed to contact with the restricting structures on a fourth main surface of the extension part when a surface opposite to the heat transfer plate is assumed as a third main surface out of the third main surface and the fourth main surface which are the front and back surfaces of the electrothermal conversion element. For example, the first high frequency electrode current line 162 functions as a second lead wire which is electrically connected to the second lead connection at the extension part and is configured to apply a high frequency voltage to the heat transfer plate.

In the first electrode part 100 according to the present exemplary embodiment, the electrothermal conversion element 140 on which are formed the first lead connections 146 and the second lead connection 148 is provided to protrude from the first high frequency electrode 110 and the cover member 150. Further, the first lead connections 146 are arranged on the first high frequency electrode 110 side of the substrate 142. Herein, the first high frequency electrode 110 is relatively thick among the components in the first electrode part 100. As illustrated in FIG. 14, the first heater current lines 164 are connected to the first lead connections 146 to extend from the electrothermal conversion element 140. Due to the fact, according to the present exemplary embodiment, the first heater current lines 164 do not contribute to the thickness of the first electrode part 100 and the first electrode part 100 is realized to be thinner (lower in its height). Similarly, the second lead connection 148 is arranged on the cover member 150 side of the substrate 142. Herein, the cover member 150 is relatively thick among the components in the first electrode part 100. As illustrated in FIG. 14, the first high frequency electrode current line 162 is connected to the second lead connection 148 to extend from the electrothermal conversion element 140. Due to the fact, according to the present exemplary embodiment, the first high frequency electrode current line 162 does not contribute to the thickness of the first electrode part 100 and the first electrode part 100 is realized to be thinner (lower in its height).

In the present exemplary embodiment, the electrothermal conversion element 140 is inserted into the gap 126 between the electrode bottom 111 and the restricting structures 114 of the first high frequency electrode 110. A force is applied to the extension parts 143 of the electrothermal conversion element 140 in a direction perpendicular to the extension parts 143. The electrothermal conversion element 140 is restricted by the restricting structures 114 and thus is prevented from releasing from the electrode bottom 111.

If the restricting structures 114 are not present, the electrothermal conversion element 140 is fixed on the first high frequency electrode 110 only by an adhesive force of the highly heat-conductive adhesive sheet 130. At this time, the electrothermal conversion element 140 can be released from the first high frequency electrode 110 due to the structure of the first high frequency electrode 110 and the limited adhesive performance of the highly heat-conductive adhesive sheet 130. If such release is caused, the first high frequency electrode 110 cannot be uniformly heated by the electrothermal conversion element 140. In the present exemplary embodiment, the restricting structures 114 are present thereby to prevent the first high frequency electrode 110 and the electrothermal conversion element 140 from releasing from each other, which prevents a non-uniform temperature on heating.

Further, in the present exemplary embodiment, the first high frequency electrode current line 162 is not directly connected to the first high frequency electrode 110, but is connected to the first high frequency electrode 110 via the second lead connection 148 and the restricting structure 114. Therefore, a flow of heat from the first high frequency electrode 110 to the first high frequency electrode current line 162 can be restricted. Consequently, a reduction in temperature of the first high frequency electrode 110 at the connection portion with the first high frequency electrode current line 162 can be restricted, which enhances a thermal efficiency in heating a body tissue.

### Second Exemplary Embodiment

A second exemplary embodiment will be described. Herein, the differences from the first exemplary embodiment will be described, and the same parts are denoted with the same reference numerals and the description thereof will be omitted. In the present exemplary embodiment, the shapes of the first high frequency electrode 110 and the second high frequency electrode 210 are different from those of the first exemplary embodiment.

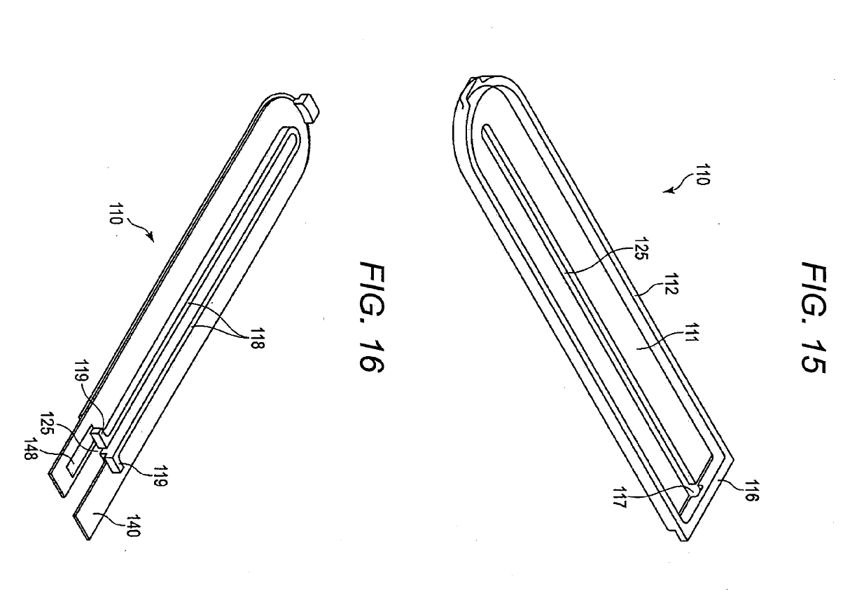

The shape of the first high frequency electrode 110 according to the present exemplary embodiment is illustrated in FIG. 15. As illustrated in FIG. 15, the first high frequency electrode 110 according to the present exemplary embodiment comprises a restricting structure 116 where the two restricting structures 114 symmetrically formed across the cutout 125 forming the first cutter guide groove 332 are coupled in the first exemplary embodiment. The restricting structure 116 is formed with a groove 117 through which the cutter 345 passes.

Other components are the same as those of the first exemplary embodiment. The present exemplary embodiment also functions like the first exemplary embodiment, and can obtain the same advantages.

The shape of the first high frequency electrode 110 may be configured such that the sidewall 112 of the first high frequency electrode 110 according to the first exemplary embodiment is not provided and an inner wall 118 is instead provided on the periphery of the cutout 125 and restricting structures 119 are provided on the inner wall 118 as illustrated in FIG. 16. Further, the first high frequency electrode 110 may include both of the sidewall 112 provided on the outer periphery of the first high frequency electrode and the inner wall 118. In this case, the shape of the cover member 150 is different from that of the first exemplary embodiment, and may be changed as needed.

Other components are the same as those of the first exemplary embodiment, and the present exemplary embodiment functions like the first exemplary embodiment and can obtain the same advantages.

1. A treatment apparatus comprising:
an electrothermal conversion element comprising:
a substrate having a first part and a second part; and
an electric resistance pattern arranged to at least the first part of the substrate, wherein the electric resistance pattern is configured to convert a first electric energy to heat;
a heat transfer plate having:
a first surface configured to contact a body tissue; and
a second surface thermally coupled with the electrothermal conversion element,
wherein the heat transfer plate is configured to conduct the heat from the second surface to the first surface, and
wherein the second part of the substrate extends past and away from an end of the heat transfer plate; and
a restricting structure arranged to be spaced apart from the second surface of the heat transfer plate,
wherein the restricting structure is configured to restrict a movement of the first part of the substrate in a direction away from the second surface of the heat transfer plate.

2. The treatment apparatus according to claim 1,
wherein the electrothermal conversion element further comprises a first lead connection arranged to at least the second part of the substrate, and
wherein the first lead connection is electrically connected to the electric resistance pattern to conduct the first electric energy to the electric resistance pattern.

3. The treatment apparatus according to claim 1, wherein the restricting structure is arranged to a base end of the heat transfer plate.

4. The treatment apparatus according to claim 1, wherein the restricting structure is configured to exert a force against the second part of the substrate of the electrothermal conversion element to restrict the movement of the first part of the substrate in the direction away from the second surface of the heat transfer plate.

5. The treatment apparatus according to claim 1, further comprising:
an adhesive sheet configured to adhere the electrothermal conversion element to the second surface of the heat transfer plate,
wherein the adhesive sheet is configured to conduct the heat from the electrothermal conversion element to the heat transfer plate.

6. The treatment apparatus according to claim 5,
wherein the electrothermal conversion element further comprises a first lead connection arranged to the second part of the substrate extending past and away from the end of the heat transfer plate,
wherein the first lead connection is electrically conductive and is electrically connected to the electric resistance pattern to apply the first electric energy to the electric resistance pattern,
wherein the adhesive sheet has an electric insulation property, and
wherein the adhesive sheet extends past and away from the end of the heat transfer plate to insulate the first lead connection from the heat transfer plate.

7. The treatment apparatus according to claim 5, wherein the adhesive sheet is arranged to adhere the electrothermal conversion element to at least the second surface of the heat transfer plate at the end of the heat transfer plate.

8. The treatment apparatus according to claim 1,
wherein the electrothermal conversion element further comprises a first lead connection arranged to the second part of the substrate extending past and away from the end of the heat transfer plate, and
wherein the first lead connection is electrically conductive and is electrically connected to the electric resistance pattern to apply the first electric energy to the electric resistance pattern.

9. The treatment apparatus according to claim 1, further comprising:
a second lead connection arranged to the second part of the substrate extending past and away from the end of the heat transfer plate,
wherein the first surface of the heat transfer plate is electrically conductive, and
wherein the restricting structure is electrically conductive and electrically connects the second lead connection and the first surface of the heat transfer plate to apply a second electric energy from the second lead connection to the first surface of the heat transfer plate.

10. The treatment apparatus according to claim 1, further comprising:
a cover configured to engage the heat transfer plate to cover the electrothermal conversion element.
